(19) United States
(12) Patent Application Publication
(10) Pub. No.: US 2026/0115210 A1
(43) Pub. Date: Apr. 30, 2026

(54) METHOD FOR TREATING SUBJECTS SUFFERING FROM CENTRAL NERVOUS SYSTEM CONTUSIONS

(71) Applicant: REMEDY PHARMACEUTICALS, INC., New York, NY (US)

(72) Inventors: Jacob SB ELKINS, Brookline, MA (US); Sven Martin JACOBSON, New York, NY (US)

(73) Assignee: REMEDY PHARMACEUTICALS, INC., New York, NY (US)

(21) Appl. No.: 19/431,340

(22) Filed: Dec. 23, 2025

Related U.S. Application Data
(63) Continuation of application No. 17/607,619.
(63) Continuation-in-part of application No. 18/595,205, filed on Mar. 4, 2024.
Application No. 18/595,205 is a continuation of application No. 17/488,591, filed on Sep. 29, 2021, now Pat. No. 11,951,085.
Application No. 17/488,591 is a division of application No. 15/763,932, now abandoned.
(60) Provisional application No. 62/238,461, filed on Oct. 7, 2015.
(60) Provisional application No. 62/842,021, filed on May 2, 2019.

(51) Int. Cl. A61K 31/64; A61K 31/047; A61P 7/04

(52) U.S. Cl. CPC A61K 31/64; A61K 31/047; A61P 7/04

(57) ABSTRACT

Methods of inhibiting expansion of cerebral contusion, inhibiting secondary hemorrhage and capillary fragmentation in the brain, reducing pericontusional edema and hemorrhage size in the brain, decreasing water content in a CNS tissue, inhibiting disruption of the blood-brain barrier, inhibiting CNS contusion progression and improving post-contusion motor function, inhibiting microvascular impairment caused by endothelial cell swelling and fragmentation, inhibiting extravasation of blood into the brain parenchyma of a subject, inhibiting endothelial cell breakdown in a brain tissue, inhibiting extravasation of endovascular fluids into the brain's interstitium, decreasing vasogenic edema as measured by T2 flair magnetic resonance imaging, and decreasing matrix metalloprotease concentration in a CNS tissue, by administering a SUR1-TRPM4 channel inhibitor alone or in combination with one or more drugs or agents.

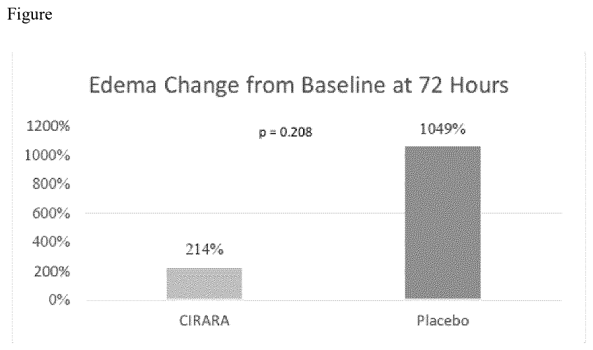

### CROSS-REFERENCE TO RELATED APPLICATIONS

This application is a continuation of U.S. application Ser. No. 17/607,619 filed Oct. 29, 2021, which claims the benefit of 35 U.S.C. 371 National Phase Entry Application from PCT/US2020/030620, filed Apr. 30, 2020, which claims the benefit of U.S. Provisional Patent Application No. 62/842,021 filed May 2, 2019, the disclosures of which are incorporated herein in their entireties by reference. This application is a continuation-in-part of U.S. Ser. No. 18/595,205 filed Mar. 4, 2024, which is a continuation of U.S. application Ser. No. 17/488,591 filed Sep. 29, 2021, granted as U.S. Pat. No. 11,951,085, which is a divisional of U.S. application Ser. No. 15/763,932 filed Nov. 8, 2018, which claims the benefit of 35 U.S.C. 371 National Phase Entry Application from PCT/US2016/055988, filed Oct. 7, 2016, which claims the benefit of U.S. Provisional Patent Application No. 62/238,461 filed Oct. 7, 2015, the disclosures of which are incorporated herein in their entireties by reference.

### FIELD OF THE DISCLOSURE

This disclosure relates to methods and materials for treating subjects suffering from central nervous system contusions.

### BACKGROUND

Central nervous system (CNS) contusion is a devastating public health problem with no approved pharmacologic treatment to stop or slow the progression of the lesion following the initial trauma. Delayed progression of bleeding is known as the hemorrhagic progression of contusion (HPC). This expansion leads not only to an increase in hematoma volume, but also to the evolution of perihematomal edema surrounding the lesion itself. After initial trauma, increased osmotic flux leads to the lysis of endothelial cells and the loss of capillary structural integrity. This results in the extravasation of blood, and the resultant expansion of the initial contusion. Moreover, loss of capillary structure permits the development of perihematomal vasogenic edema. Further, cytotoxic edema is another sequela that worsens contusion progression. Such sequelae cause significant secondary brain injury. The prognosis for patients with HPC is poor, with case fatality in a majority of cases.

CNS contusion often causes significant long-term morbidity and mortality. Bleeding into the brain parenchyma is problematic, due not only to the mass effect of a hematoma on intracranial pressure and associated brain herniation with compression of vital neurologic centers, but also to the neurotoxicity of hemoglobin. In addition to the initial bleed following trauma, contusions often progress in size during the acute hospitalization period, significantly worsening prognosis.

Progression of contusions is largely a function of the initial contusion volume, although other factors also contribute. Use of traditional and novel anticoagulants, antiplatelet agents, coagulopathy, age, and uncontrolled hypertension are all thought to contribute to some degree to progression and are thus controlled, if possible. Other processes that contribute to the progression of a contusion are analogous to the dysfunction of the cerebral microvasculature. Microvascular impairment, caused primarily by endothelial cell swelling and fragmentation, worsens hematoma expansion via the ongoing extravasation of blood into the brain parenchyma. In addition to ongoing bleeding, endothelial cell breakdown also permits the formation of perihematomal edema by extravasation of endovascular fluids into the brain's interstitium.

There is no approved pharmacologic treatment to stop or slow the progression of CNS contusions. The standard of care remains primarily supportive, with the maintenance of hemostasis to limit ongoing bleeding, and with surgical evacuation or other means to reduce intracranial pressure (i.e., insertion of ventricular drains, osmotherapy, or decompressive craniectomy [DC]). Despite these interventions, CNS contusions often continue to progress, particularly during the initial 12 hours. There is no standard of care to limit expansion of brain contusion, leaving an urgent unmet medical need. Studies of hemostatic agents, including recombinant factor 7 and tranexamic acid, have yielded mixed results and are not routinely used in clinical practice. Therefore, there is a need to provide treatments to stop or slow the progression of CNS contusions.

### SUMMARY OF THE INVENTION

The present disclosure includes methods of inhibiting expansion of cerebral contusions by administering a SUR1-TRPM4 channel inhibitor alone or in combination with one or more drugs or agents to a subject according to a specific dosing regimen to start within a predetermined time of initial contusion to the brain, spinal cord or other part of the CNS. In certain aspects, the SUR1-TRPM4 channel inhibitor is glyburide, a pharmaceutically acceptable salt thereof, variant or derivative thereof

In a first aspect, the present disclosure provides methods of inhibiting expansion of cerebral contusions by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a second aspect, the present disclosure provides a method for inhibiting secondary hemorrhage and capillary fragmentation in the brain of the subject, by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a third aspect, the present disclosure provides a method for reducing pericontusional edema and hemorrhage size by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a fourth aspect, the present disclosure provides a method for decreasing water content in a CNS tissue after CNS contusion by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a fifth aspect, the present disclosure provides a method for inhibiting disruption of the blood-brain barrier after a CNS contusion, by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a sixth aspect, the present disclosure provides a method for inhibiting CNS contusion progression and improving post-contusion motor function, by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a seventh aspect, the present disclosure provides a method for inhibiting microvascular impairment caused by endothelial cell swelling and fragmentation, by administering a SUR1-TRPM4 channel inhibitor alone or in combination with one or more drugs or agents to a subject according to a specific dosing regimen to start within a predetermined time of initial contusion to the brain, spinal cord or other part of the CNS.

In an eighth aspect, the present disclosure provides a method for inhibiting extravasation of blood into the brain parenchyma, by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a ninth aspect, the present disclosure provides a method for inhibiting endothelial cell breakdown in a brain tissue, by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a tenth aspect, the present disclosure provides a method for inhibiting extravasation of endovascular fluids into the brain's interstitium, by administering a SUR1-TRPM4 channel inhibitor alone or in combination with one or more drugs or agents to a subject according to a specific dosing regimen to start within a predetermined time of initial contusion to the brain, spinal cord or other part of the CNS.

Other features and characteristics of the subject matter of this disclosure, as well as the methods of operation, functions of related elements of structure and the combination of parts, and economies of manufacture, will become more apparent upon consideration of the following description and the appended claims, all of which form a part of this specification.

### BRIEF DESCRIPTION OF THE DRAWING

The FIGURE is a graph showing the % change in edema at 72 hours compared to baseline for patients with traumatic brain injury administered glyburide (CIRARA) or placebo.

### DETAILED DESCRIPTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or embodiments so described.

The singular forms “a,” “an,” and “the” include plural referents unless the context clearly dictates otherwise.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, delaying or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. In addition to being useful as methods of treatment, the methods described herein may be useful for the prevention or prophylaxis of disease.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” would find direct support due to this definition.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein. For example, in one aspect, the degree of flexibility can be within about ±10% of the numerical value. In another aspect, the degree of flexibility can be within about ±5% of the numerical value. In a further aspect, the degree of flexibility can be within about ±2%, ±1%, or ±0.05%, of the numerical value.

Generally herein, the term “or” includes “and/or.”

As used herein, a plurality of compounds or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Furthermore, certain compositions, injuries or conditions, steps, or the like may be discussed in the context of one specific embodiment or aspect. It is understood that this is merely for convenience, and such disclosure is equally applicable to other embodiments and aspects found herein. For example, a list of method steps, active agents, kits or compositions described with respect to a method of treating brain contusion would find direct support for embodiments related to methods of reducing capillary fragmentation or reducing extravasation of blood into the brain, even if those method steps, active agents, kits or compositions are not re-listed in the context of that embodiment in the specification.

In a first aspect, the present disclosure provides methods of inhibiting expansion of cerebral contusions by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a second aspect, the present disclosure provides a method for inhibiting secondary hemorrhage and capillary fragmentation in the brain of the subject, by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a third aspect, the present disclosure provides a method for reducing pericontusional edema and hemorrhage size by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a fourth aspect, the present disclosure provides a method for decreasing water content in a CNS tissue after CNS contusion by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a fifth aspect, the present disclosure provides a method for inhibiting disruption of the blood-brain barrier after a CNS contusion, by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a sixth aspect, the present disclosure provides a method for inhibiting CNS contusion progression and improving post-contusion motor function, by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a seventh aspect, the present disclosure provides a method for inhibiting microvascular impairment caused by endothelial cell swelling and fragmentation, by administering a SUR1-TRPM4 channel inhibitor alone or in combination with one or more drugs or agents to a subject according to a specific dosing regimen to start within a predetermined time of initial contusion to the brain, spinal cord or other part of the CNS.

In an eighth aspect, the present disclosure provides a method for inhibiting extravasation of blood into the brain parenchyma, by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a ninth aspect, the present disclosure provides a method for inhibiting endothelial cell breakdown in a brain tissue, by administering a SUR1-TRPM4 channel inhibitor alone or in combination with one or more drugs or agents to a subject according to a specific dosing regimen to start within a predetermined time of initial contusion to the brain, spinal cord or other part of the CNS.

In a tenth aspect, the present disclosure provides a method for inhibiting extravasation of endovascular fluids into the brain's interstitium, by administering a SUR1-TRPM4 channel inhibitor alone or in combination with one or more drugs or agents to a subject according to a specific dosing regimen to start within a predetermined time of initial contusion to the brain, spinal cord or other part of the CNS.

As used herein and in relation to every disclosed aspect of the present disclosure, a SUR1-TRPM4 channel inhibitor may include glyburide, 4-trans-hydroxy-glibenclamide, 3-cis-hydroxyglibenclamide, tolbutamide, chlorpropamide, tolazamide, repaglinide, nateglinide, meglitinide, midaglizole, tolazamide, gliquidone, LY397364, LY389382, gliclazide, glimepiride, metabolites that interact with SUR1, salts, and combinations thereof.

As used herein and in relation to every disclosed aspect of the present disclosure, glyburide variants and derivatives may include but are not limited to 4-trans-hydroxy-glibenclamide, 3-cis-hydroxyglibenclamide, tolbutamide, chlorpropamide, tolazamide, repaglinide, nateglinide, meglitinide, midaglizole, gliquidone, LY397364, LY389382, gliclazide, glimepiride. In certain aspects, including the first through tenth aspects, the specific dosing regimen involves administering glyburide, a pharmaceutically acceptable salt thereof, variant or derivative thereof in an amount, at a rate, and for a duration to maintain a systemic steady state plasma level in a range of about 20-40 ng/mL, about 22-36 ng/mL, about 24-34 ng/mL, about 26-32 ng/mL, about 28-30 ng/mL, or about 25 ng/ml, about 26 ng/ml, about 27 ng/mL, about 28 ng/ml, about 29 ng/ml, about 30 ng/ml, about 31 ng/mL, or about 32 ng/mL, for a contusion-inhibiting treatment period. It has been found that the specific dosing regimen of the present disclosure is critical for achieving a therapeutic effect of sufficiently inhibiting expansion of CNS contusion in human subjects suffering from CNS contusion expansion, without causing dose-limiting side effects in those subjects.

In IV-treated subjects, e.g., humans, glyburide exhibits two-compartment pharmacokinetics such that after bolus injection, its plasma concentration undergoes rapid decline. This rapid decline is problematic because it is critical to maintain target plasma levels of glyburide for the duration of the therapy to inhibit CNS contusion expansion, particularly during the initial 6 hour period. CNS contusion expansion has a distinct pathophysiology from large hemispheric infarction and cytotoxic edema. Unlike large hemispheric infarction and cytotoxic edema treatments that involve 72-hour infusion periods, it is critical to treat patients suffering from brain contusion expansion for a contusion-inhibiting treatment period of 96 hours. In order to achieve and maintain target plasma levels of glyburide for the duration of the therapy over the course of 96 hours, the present disclosure provides a specific dosing regimen involving a bolus injection of glyburide, a pharmaceutically acceptable salt thereof, variant or derivative thereof, followed by a high dose continuous infusion of glyburide, a pharmaceutically acceptable salt thereof, variant or derivative thereof, and then a low dose continuous infusion of glyburide, a pharmaceutically acceptable salt thereof, variant or derivative thereof, that is maintained for 90 hours.

The present disclosure provides a method of inhibiting expansion of cerebral contusions by administering an amount and rate of administration of glyburide, a pharmaceutically acceptable salt thereof, variant or derivative thereof, during the bolus phase and the high dose continuous infusion phase that achieve and maintain target plasma levels of glyburide during the critical first hours after the CNS contusion to effectively inhibit contusion expansion during a period when patients are at highest risk for contusion expansion. In certain aspects, including the first through tenth aspects, an additional bolus may be administered after the initial bolus.

In certain aspects, including the first through tenth aspects, the present disclosure provides a method of inhibiting expansion of cerebral contusions by administering an active agent within a predetermined time of initial contusion to the brain, spinal cord or other part of the CNS. In one aspect, the first administration of the active agent may be within the first hour, first 2 hours, first 3 hours, first four 4 hours, first 6 hours, first 8 hours, or first 10 hours of the initial contusion to the brain, spinal cord or other part of the CNS.

In certain aspects, including the first through tenth aspects, the present disclosure provides a method of inhibiting expansion of cerebral contusions by administering about 3 mg to about 5.5 mg glyburide, a pharmaceutically acceptable salt thereof, variant or derivative thereof, on day 1, about 2.5 mg to about 5 mg glyburide, a pharmaceutically acceptable salt thereof, variant or derivative thereof, on day 2, about 2.5 mg to about 5 mg glyburide, a pharmaceutically acceptable salt thereof, variant or derivative thereof, on day 3, and about 2.5 mg to about 5 mg glyburide, a pharmaceutically acceptable salt thereof, variant or derivative thereof, on day 4. In certain aspects, including the first through tenth aspects, the present disclosure provides a method of inhibiting expansion of cerebral contusions by administering about 3-3.5 mg glyburide, a pharmaceutically acceptable salt thereof, variant or derivative thereof, on day 1, and about 2.5-2.8 mg glyburide, a pharmaceutically acceptable salt thereof, variant or derivative thereof, on days 2-4. In certain aspects, including the first through tenth aspects, the present disclosure provides a method of inhibiting expansion of cerebral contusions by administering about 5-5.5 mg glyburide, a pharmaceutically acceptable salt thereof, variant or derivative thereof, on day 1, and about 4.2-4.8 mg glyburide, a pharmaceutically acceptable salt thereof, variant or derivative thereof, on days 2-4. In certain aspects, including the first through tenth aspects, the present disclosure provides a method of inhibiting expansion of cerebral contusions by administering about 3.2 mg glyburide, a pharmaceutically acceptable salt thereof, variant or derivative thereof, on day 1, and about 2.7 mg glyburide, a pharmaceutically acceptable salt thereof, variant or derivative thereof, on days 2-4. In certain aspects, including the first through tenth aspects, the present disclosure provides a method of inhibiting expansion of cerebral contusions by administering about 5.2 mg glyburide, a pharmaceutically acceptable salt thereof, variant or derivative thereof, on day 1, and about 4.5 mg glyburide, a pharmaceutically acceptable salt thereof, variant or derivative thereof, on days 2-4.

In certain aspects, including the first through tenth aspects, the initial bolus injection is administered by a slow intravenous push. In certain aspects, including the first through tenth aspects, the slow intravenous push is performed over a period of about 60 to 300 seconds, about 90 to 180 seconds, or about 120 seconds. In certain aspects, including the first through tenth aspects, the initial bolus injection involves administering an aqueous solution of about 0.1 to about 0.2 mM glyburide, a pharmaceutically acceptable salt thereof, variant or derivative thereof, about 0.11 to about 0.15 mM glyburide, a pharmaceutically acceptable salt thereof, variant or derivative thereof, or about 0.11 to about 0.12 mM glyburide, a pharmaceutically acceptable salt thereof, variant or derivative thereof.

In certain aspects, including the first through tenth aspects, the initial bolus injection involves injecting a glyburide solution in a volume of about 20 to 40 mL, 22 to 38 mL, 22 to 25 mL, 35 to 40 mL, or any volume within the recited ranges. In certain aspects, including the first through tenth aspects, the initial bolus injection involves injecting a glyburide solution via a syringe or other appropriate injection device. In certain aspects, including the first through tenth aspects, the initial bolus injection involves injecting 0.1 to 0.3 mg glyburide, a pharmaceutically acceptable salt thereof, variant or derivative thereof, 0.12 to 0.25 mg glyburide, a pharmaceutically acceptable salt thereof, variant or derivative thereof, 0.13 to 0.18 mg glyburide, a pharmaceutically acceptable salt thereof, variant or derivative thereof, 0.2 to 0.24, 0.13, 0.14, 0.15, 0.16, 0.17, 0.21, 0.22, 0.23 mg glyburide, a pharmaceutically acceptable salt thereof, variant or derivative thereof, or any amount falling within the recited ranges.

In certain aspects, including the first through tenth aspects, the high dose continuous infusion is administered for a period of about 4 to about 10 hours, about 6 to about 8 hours, about 5, about 6 or about 7 hours in an amount sufficient to maintain a systemic steady state plasma level of 20-40 ng/mL, 24-35 ng/mL, 25 ng/mL, 26 ng/ml, 27 ng/ml, 28 ng/mL, 29 ng/ml, 30 ng/ml, 31 ng/mL, 32 ng/mL. In certain aspects, including the first through tenth aspects, the high dose continuous infusion involves administering an aqueous solution of about 0.001 to about 0.1 mM glyburide, a pharmaceutically acceptable salt thereof, variant or derivative thereof, about 0.005 mM to about 0.5 mM glyburide, a pharmaceutically acceptable salt thereof, variant or derivative thereof, or about 0.005 to about 0.01 mM glyburide, a pharmaceutically acceptable salt thereof, variant or derivative thereof. In certain aspects, the aqueous solution contains saline.

In certain aspects, the present disclosure includes using a vial containing a lyophilized glyburide, a pharmaceutically acceptable salt thereof, variant or derivative thereof, mannitol and a base. In certain aspects, the present disclosure includes using a syringe, IV bag, or IV tube containing a stable composition for infusion containing saline solution in which the lyophilized glyburide, a pharmaceutically acceptable salt thereof, variant or derivative thereof, mannitol, and a base has been reconstituted. In some aspects, the stable composition for infusion is stable at room temperature for 24-36 hours, 26-34 hours, 28-32, or 30 hours.

In certain aspects, the present disclosure includes a kit containing four unit dosage forms containing lyophilized glyburide, a pharmaceutically acceptable salt thereof, variant or derivative thereof, mannitol and a base, wherein each unit dosage form contains 3-8 mg glyburide. In certain aspects, each unit dosage form contains 3, 5, or 6 mg glyburide. In certain aspects, each unit dosage form is a vial. In certain aspects, each kit contains instructions specifically instructing a user to perform one or more steps provided in the present disclosure.

In certain aspects, including the first through tenth aspects, the high dose continuous infusion involves administering about 0.8 to about 2 mg glyburide, a pharmaceutically acceptable salt thereof, variant or derivative thereof, about 0.9 to about 1.7 mg glyburide, a pharmaceutically acceptable salt thereof, variant or derivative thereof, about 1 to about 1.7 mg glyburide, a pharmaceutically acceptable salt thereof, variant or derivative thereof, about 1 mg glyburide, a pharmaceutically acceptable salt thereof, variant or derivative thereof, or about 1.6 mg glyburide, a pharmaceutically acceptable salt thereof, variant or derivative thereof, or any amount falling within the recited ranges. In certain aspects, including the first through tenth aspects, the high dose continuous infusion involves administering glyburide, a pharmaceutically acceptable salt thereof, variant or derivative thereof at a rate of about 0.15 mg per hour to about 0.3 mg per hour, about 0.16 mg per hour to about 0.28 mg per hour, about 0.16 mg per hour to about 0.18 mg per hour, about 0.25 mg per hour to about 0.28 mg per hour, about 0.164 mg per hour, about 0.272 mg per hour, or any rate falling within the recited ranges.

In certain aspects, including the first through tenth aspects, the method of the present disclosure includes administering glyburide, a pharmaceutically acceptable salt thereof, variant or derivative thereof in a low dose infusion in an amount of about 8- to 12-fold higher than the amount of glyburide, a pharmaceutically acceptable salt thereof, variant or derivative thereof administered in a high dose infusion. In certain aspects, including the first through tenth aspects, the method of the present disclosure includes administering glyburide, a pharmaceutically acceptable salt thereof, variant or derivative thereof in a low dose infusion in an amount of about 10-fold higher than the amount of glyburide, a pharmaceutically acceptable salt thereof, variant or derivative thereof administered in a high dose infusion. In certain aspects, including the first through tenth aspects, the method of the present disclosure includes administering glyburide, a pharmaceutically acceptable salt thereof, variant or derivative thereof, in a low dose infusion in an amount of about 75- to 100-fold higher than the amount of glyburide, a pharmaceutically acceptable salt thereof, variant or derivative thereof administered in the bolus phase. In certain aspects, including the first through tenth aspects, the method of the present disclosure includes administering glyburide, a pharmaceutically acceptable salt thereof, variant or derivative thereof, in a low dose infusion in an amount of about 78- to 80-fold higher than the amount of glyburide, a pharmaceutically acceptable salt thereof, variant or derivative thereof administered in the bolus phase. In certain aspects, including the first through tenth aspects, the method of the present disclosure includes administering glyburide, a pharmaceutically acceptable salt thereof, variant or derivative thereof, in a high dose infusion in an amount of about 7- to 10-fold higher than the amount of glyburide, a pharmaceutically acceptable salt thereof, variant or derivative thereof administered in the bolus phase. In certain aspects, including the first through tenth aspects, the method of the present disclosure includes administering glyburide, a pharmaceutically acceptable salt thereof, variant or derivative thereof, in a high dose infusion in an amount of about 7.5- to 8-fold higher than the amount of glyburide, a pharmaceutically acceptable salt thereof, variant or derivative thereof administered in the bolus phase.

In certain aspects, including the first through tenth aspects, the method of the present disclosure includes administering glyburide, a pharmaceutically acceptable salt thereof, variant or derivative thereof, in a weight ratio of about 0.1-0.13:0.9-1.1:10-10.5 between the bolus phase, the high dose continuous infusion phase, and the low dose continuous infusion phase, respectively.

In certain aspects, including the first through tenth aspects, the method of the present disclosure includes administering glyburide, a pharmaceutically acceptable salt thereof, variant or derivative thereof in a low dose infusion rate of about 20- to 25-fold lower than the amount of glyburide, a pharmaceutically acceptable salt thereof, variant or derivative thereof administered in the bolus phase. In certain aspects, including the first through tenth aspects, the method of the present disclosure includes administering glyburide, a pharmaceutically acceptable salt thereof, variant or derivative thereof in a low dose infusion rate of about 23- to 24-fold lower than the amount of glyburide, a pharmaceutically acceptable salt thereof, variant or derivative thereof administered in the bolus phase. In certain aspects, including the first through tenth aspects, the method of the present disclosure includes administering glyburide, a pharmaceutically acceptable salt thereof, variant or derivative thereof in a low dose infusion rate of about 1.2- to 1.5-fold lower than the amount of glyburide, a pharmaceutically acceptable salt thereof, variant or derivative thereof administered in the high dose continuous infusion phase. In certain aspects, including the first through tenth aspects, the method of the present disclosure includes administering glyburide, a pharmaceutically acceptable salt thereof, variant or derivative thereof in a low dose infusion rate of about 1.4 to 1.5-fold lower than the amount of glyburide, a pharmaceutically acceptable salt thereof, variant or derivative thereof administered in the high dose continuous infusion phase. In certain aspects, including the first through tenth aspects, the method of the present disclosure includes administering glyburide, a pharmaceutically acceptable salt thereof, variant or derivative thereof in a high dose infusion rate of about 23 to 24-fold lower than the amount of glyburide, a pharmaceutically acceptable salt thereof, variant or derivative thereof administered in the bolus phase.

In certain aspects, including the first through tenth aspects, the method of the present disclosure includes administering glyburide, a pharmaceutically acceptable salt thereof, variant or derivative thereof, in an hourly administration rate ratio of about 3.0-4.0:0.12-0.18:0.1-0.15 between the bolus phase, the high dose continuous infusion phase, and the low dose continuous infusion phase, respectively.

In certain aspects, including the first through tenth aspects, the low dose continuous infusion is administered for a period of about 80 to about 120 hours, about 85 to about 100 hours, about 88, about 90 or about 92 hours in an amount sufficient to maintain a systemic steady state plasma level of 20-35 ng/mL, 24-33 ng/mL, 25 ng/mL, 26 ng/mL, 27 ng/mL, 28 ng/ml, 29 ng/ml, 30 ng/ml, 31 ng/mL, 32 ng/mL. In certain aspects, including the first through tenth aspects, the low dose continuous infusion involves administering an aqueous solution of about 0.001 to about 0.1 mM glyburide, a pharmaceutically acceptable salt thereof, variant or derivative thereof, about 0.005 mM to about 0.5 mM glyburide, a pharmaceutically acceptable salt thereof, variant or derivative thereof, or about 0.005 to about 0.01 mM glyburide, a pharmaceutically acceptable salt thereof, variant or derivative thereof. In certain aspects, including the first through tenth aspects, the low dose continuous infusion involves administering about 8 to about 20 mg glyburide, a pharmaceutically acceptable salt thereof, variant or derivative thereof, about 10 to about 17 mg glyburide, a pharmaceutically acceptable salt thereof, variant or derivative thereof, about 10 mg glyburide, a pharmaceutically acceptable salt thereof, variant or derivative thereof, or about 17 mg glyburide, a pharmaceutically acceptable salt thereof, variant or derivative thereof, or any amount falling within the recited ranges. In certain aspects, including the first through tenth aspects, the low dose continuous infusion involves administering glyburide, a pharmaceutically acceptable salt thereof, variant or derivative thereof at a rate of about 0.10 mg per hour to about 0.2 mg per hour, about 0.11 mg per hour to about 0.19 mg per hour, about 0.11 mg per hour to about 0.12 mg per hour, about 0.18 mg per hour to about 0.19 mg per hour, about 0.113 mg per hour, about 0.187 mg per hour, or any rate falling within the recited ranges.

In certain aspects, including the first through tenth aspects, the method of the present disclosure includes a total treatment course of 10-20 mg glyburide, a pharmaceutically acceptable salt thereof, variant or derivative thereof, during a period of highest risk for contusion expansion. In certain aspects, including the first through tenth aspects, the method of the present disclosure includes a total treatment course of about 11-14 mg glyburide, a pharmaceutically acceptable salt thereof, variant or derivative thereof, over 96 hours. In certain aspects, including the first through tenth aspects, the method of the present disclosure includes a total treatment course of about 17-20 mg glyburide, a pharmaceutically acceptable salt thereof, variant or derivative thereof, over 96 hours. In certain aspects, including the first through tenth aspects, the method of the present disclosure includes a total treatment course of about 11 mg glyburide, a pharmaceutically acceptable salt thereof, variant or derivative thereof, over 96 hours. In certain aspects, including the first through tenth aspects, the method of the present disclosure includes a total treatment course of about 19 mg glyburide, a pharmaceutically acceptable salt thereof, variant or derivative thereof, over 96 hours.

In certain aspects, including the first through tenth aspects, the present disclosure includes a method for decreasing vasogenic edema as measured by T2 flair magnetic resonance imaging comprising administering about 18-20 mg glyburide, a pharmaceutically acceptable salt thereof, variant or derivative thereof, over 96 hours, including a bolus injection of at least 5 mg glyburide, a pharmaceutically acceptable salt thereof, variant or derivative thereof, a systemic steady state plasma level of 32-40 ng/mL, and continuous infusions at hourly infusion rate ratios of 1.2 to 1.7, 1.3 to 1.6, or 1.4 to 1.5 for the high dose infusion to the low dose infusion, wherein the low dose infusion is administered for 80 to 94 hours, 86 to 92 hours, or 90 hours.

In certain aspects, including the first through tenth aspects, the present disclosure includes a method for decreasing matrix metalloprotease concentration in the subject comprising administering about 18-20 mg glyburide, a pharmaceutically acceptable salt thereof, variant or derivative thereof, over 96 hours, including a bolus injection of at least 5 mg glyburide, a pharmaceutically acceptable salt thereof, variant or derivative thereof, a systemic steady state plasma level of 32-40 ng/mL, and continuous infusions at hourly infusion rate ratios of 1.2 to 1.7, 1.3 to 1.6, or 1.4 to 1.5 for the high dose infusion to the low dose infusion, wherein the low dose infusion is administered for 80 to 94 hours, 86 to 92 hours, or 90 hours. In some aspects, the matrix metalloprotease is MMP-9.

In certain aspects, including the first through tenth aspects, the present disclosure includes co-administering glyburide, a pharmaceutically acceptable salt thereof, variant or derivative thereof, with a sugar, a sugar alcohol or a combination thereof at a weight ratio of 1:10 to 1:1000, 1:20 to 1:500, 1:25 to 1:100, or 1:30. In certain aspects, the sugar or sugar alcohol may include, but is not limited to allitol, arabitol, dextrose, dulcitol, erythritol, galactitol, glycol, glycerol, iditol, isomalt, lactitol, maltitol, mannitol, sorbitol, threitol, xylitol, and combinations thereof. In certain aspects, the present disclosure includes co-administering glyburide, a pharmaceutically acceptable salt thereof, variant or derivative thereof, with mannitol at a weight ratio of 1:20 to 1:100, 1:25 to 1:50, or 1:30 to 1:40.

In one aspect, the administering step of the method of the present disclosure involves administering an aqueous glyburide solution through a dedicated peripheral IV line. In certain aspects, the administering step of the method of the present disclosure involves using a calibrated infusion pump with a dedicated infusion line. In certain aspects, the present disclosure excludes administration using a central line. In certain aspects, the present disclosure excludes administration using a peripherally-inserted central catheter line. In certain aspects, the present disclosure excludes administration using a central line. In certain aspects, the present disclosure excludes administration using inline filters. In certain aspects, the present disclosure excludes administration using polyvinyl chloride bags and lines.

In one aspect, the present disclosure provides a method for treating a subject at risk for CNS contusion expansion according to the above-described aspects such that the risk of the subject exhibiting contusion expansion at hour 96 after treatment with the method of the present disclosure is at least 2- to 30-fold, 3-fold, 4-fold, 5-fold, 10-fold, 15-fold, or any quantity within the recited range, lower than the risk of a subject exhibiting contusion expansion at hour 96 without treatment with the method of the present disclosure. Contusion expansion, as used herein, is exhibited as either ≥50% increase from baseline contusion volume and ≥4.0 mL absolute increase from baseline, or ≥10.0 mL total expansion from baseline. In some aspects, the risk of a subject exhibiting contusion expansion at hour 96 without treatment with the method of the present disclosure will range between 40% and 60%, whereas the risk of a subjects exhibiting contusion expansion at hour 96 after treatment with the method of the present disclosure will range between 1% to 30%, 2% to 20%, 3% to 10%, or any percentage within the recited ranges.

In one aspect, the present disclosure provides a method for treating a subject at risk for CNS contusion expansion according to the above-described aspects, further involving obtaining one or more images of the subject's CNS tissue before, during, and/or after the treatment course. In one aspect, the baseline image is a non-contrast computed tomography (NCCT), MRI image, or both. In one aspect, the 96 hour image is a non-contrast computed tomography (NCCT), MRI image, or both. In one aspect, a non-contrast computed tomography (NCCT), MRI image, or both, are obtained at 12 hours, 24, hours, 36 hours, 48 hours, 60 hours, and/or 72 hours after initiation of the treatment method of the present disclosure. In one aspect, a non-contrast computed tomography (NCCT), MRI image, or both, are obtained after 96 hours after initiation of treatment, e.g., within days, weeks, or months after a 96-hour treatment course with glyburide, a pharmaceutically acceptable salt thereof, variant or derivative thereof. In certain aspects, if a non-contrast computed tomography (NCCT) image and/or MRI image indicates that contusion expansion has not ceased or restarted, the present disclosure includes a step of repeating the treatment method of the present disclosure.

In one aspect, the present disclosure provides a method for reducing the risk for CNS contusion expansion according to the above-described aspects such that measurements of contusion expansion. In certain aspects, the present disclosure provides a method for reducing the risk that a subject at risk for contusion expansion will, based on a comparison of baseline images and 96-hour scan images, demonstrate: either ≥50% increase from baseline contusion volume and ≥4.0 mL absolute increase from baseline, or ≥10.0 mL total expansion from baseline, compared to a subject risk for contusion expansion that is not treated with the method of the present disclosure. In other aspects, the present disclosure provides a method for reducing the risk that a subject at risk for contusion expansion will, based on a comparison of baseline images and scan images prior to neurosurgical intervention (NSx), demonstrate: either ≥50% increase from baseline contusion volume and ≥4.0 mL absolute increase from baseline, or ≥10.0 mL total expansion from baseline, compared to a subject risk for contusion expansion that is not treated with the method of the present disclosure. In other aspects, the present disclosure provides a method for reducing the risk that a subject at risk for contusion expansion will, based on a comparison of baseline images and scan images prior to comfort measures only (CMO) demonstrate: either ≥50% increase from baseline contusion volume and ≥4.0 mL absolute increase from baseline, or ≥10.0 mL total expansion from baseline, compared to a subject risk for contusion expansion that is not treated with the method of the present disclosure. In certain aspects, NSx includes craniotomy and decompressive craniectomy (DC). As used herein, baseline refers to the state of the subject's CNS tissue prior to treatment with the method of the present disclosure. As used herein, the phrase 96-hour scan images refers to images of the subject's CNS tissue after treatment with the method of the present disclosure for 96 hours.

In one aspect, the present disclosure provides a method for treating a subject at risk for CNS contusion expansion according to the above-described aspects, further involving measuring total contusion volume at baseline and after 96 hours of treatment. In some aspects, the present disclosure additionally includes measuring total contusion volume after 24, 36, and/or 72 hours of treatment. In one aspect, the total contusion volume is measured by NCCT, MRI, or both. In one aspect, the present disclosure provides a method for treating a subject at risk for CNS contusion expansion according to the above-described aspects, further involving measuring absolute hematoma volume at baseline and after 96 hours of treatment. In some aspects, the present disclosure additionally includes measuring absolute hematoma volume after 24, 36, and/or 72 hours of treatment. In one aspect, the absolute hematoma volume is measured by NCCT, MRI, or both. In one aspect, the present disclosure provides a method for treating a subject at risk for CNS contusion expansion according to the above-described aspects, further involving measuring absolute edema volume at baseline and after 96 hours of treatment. In some aspects, the present disclosure additionally includes measuring absolute edema volume after 24, 36, and/or 72 hours of treatment. In one aspect, the absolute edema volume is measured by NCCT, MRI, or both. In one aspect, the present disclosure provides a method for treating a subject at risk for CNS contusion expansion according to the above-described aspects, further involving measuring midline shift at at least one of baseline, after 24, 36, 72, or 96 hours of treatment.

In one aspect, the present disclosure provides a method for treating a subject at risk for CNS contusion expansion according to the above-described aspects, further involving measuring the subject's blood glucose level. In one aspect, the measuring step may include blood glucose monitoring hourly (±30 minutes) for Hour 0 to Hour 24, every 2 hours (±30 minutes) for Hour 25 to Hour 48, and every 4 hours (±60 minutes) for Hour 49 to Hour 96. In one aspect, the present disclosure provides a method for treating a subject at risk for CNS contusion expansion according to the above-described aspects, further involving measuring the subject's blood glucose level, and reducing the dose of the glyburide, a pharmaceutically acceptable salt thereof, variant or derivative thereof, if the subject's blood glucose level is <55 mg/dL (˜3.0 mmol/L). In certain aspects, if the subject's blood glucose level is <55 mg/dL (˜3.0 mmol/L), the rate of glyburide, a pharmaceutically acceptable salt thereof, variant or derivative thereof, administered per hour is reduced by 25-75%, 30-70%, or any percentage within the recited ranges. In certain aspects, if the subject's blood glucose level is <55 mg/dL (˜3.0 mmol/L), the rate of glyburide, a pharmaceutically acceptable salt thereof, variant or derivative thereof, administered per hour is 0.05 to 0.10 mg/hr.

In one aspect, the present disclosure provides a method for treating a subject at risk for CNS contusion expansion according to the above-described aspects, measuring the subject's blood glucose level, reducing the dose of glyburide, a pharmaceutically acceptable salt thereof, variant or derivative thereof, if the subject's blood glucose level is <55 mg/dL (˜3.0 mmol/L), and subsequently re-testing the subject's blood glucose level, and increasing the dose of glyburide, a pharmaceutically acceptable salt thereof, variant or derivative thereof, if the subject's blood glucose level is <80 mg/dL (˜4.4 mmol/L). In certain aspects, if the subject's blood glucose level is <80 mg/dL for three consecutive readings, the rate of glyburide, a pharmaceutically acceptable salt thereof, variant or derivative thereof, administered per hour is increased by 15-65%, 20-55%, 30-50% or any percentage within the recited ranges, above the dose rate administered when the patient had a blood glucose level of <55 mg/dL. In certain aspects, if the subject's blood glucose level is <80 mg/dL, the rate of glyburide, a pharmaceutically acceptable salt thereof, variant or derivative thereof, administered per hour is 0.10-0.15 mg/hr.

In one aspect, the present disclosure provides a method for treating a subject at risk for CNS contusion expansion according to the above-described aspects, measuring the subject's blood glucose level, and administering a dextrose solution if the blood glucose level of the subject is about 100 mg/dL or less. In another embodiment, the dextrose solution is administered initially if the blood glucose level in the subject is about 95 mg/dL or less. In yet another embodiment, the dextrose solution is administered initially if the blood glucose levels in the subject are about 90 mg/dL or less. In yet another embodiment, the dextrose solution is administered initially if the blood glucose levels in the subject are about 80 mg/dL or less. In a further embodiment, the dextrose solution is administered initially if the blood glucose levels in the subject are about 110 mg/dL or less. In a further embodiment, the dextrose solution is administered initially if the blood glucose levels in the subject are about 120 mg/dL or less. In yet another embodiment, a dextrose solution is administered if there is a rapid downward trend in the subject's blood glucose levels. Generally, if blood glucose is greater than about 140 mg/dL, dextrose supplementation should not typically be performed. A rapid downward trend would be defined by the clinician, but could include a reduction of ≥10 mg/dl, ≥20 mg/dL, ≥30 mg/dL, ≥40 mg/dL, ≥50 mg/dl, ≥60 mg/dl, ≥70 mg/dL, ≥80 mg/dL, ≥90 mg/dL, or ≥100 mg/dL since the last measurement.

The dextrose solution can be included of dextrose in saline or in water. The weight percentage of the dextrose in the dextrose solution typically be from 1 wt % to 25 wt %, though concentrations outside of this range can also be used, e.g., 2 wt % to 20 wt %, 3 wt % to 15 wt %, 3 wt % to 12 wt %, 3 wt % to 8 wt %, 8 wt % to 12 wt %, etc. In other words, the weight percentage of the dextrose in the dextrose solution can vary and can be administered to the subject based on certain variables, including the subject's blood glucose levels. Exemplary dextrose solutions such as these that are commonly used can include 5 wt % dextrose in normal saline (DSNS) and 10 wt % dextrose in normal saline (D10NS), but such solutions may also be in water or some fraction the normal saline (e.g., ½ normal saline) rather than normal saline. In one embodiment, when the subject's blood glucose level is from greater than about 80 mg/dL to 100 mg/dL, a 3 wt % to 8 wt % solution of dextrose can be administered to the subject at from about 50 cc/hr to about 120 cc/hr. In another embodiment, when the subject's blood glucose level is from about 55 mg/dL to 80 mg/dL, an 8 wt % to 12 wt % solution of dextrose can be administered to the subject at from 50 cc/hr to 120 cc/hr. In yet another embodiment, when the subject's blood glucose level is <55 mg/dL, an 8 wt % to 12 wt % solution of dextrose can be administered to the subject at from 50 cc/hr to 120 cc/hr. In a further embodiment, when the subject's blood glucose level falls below 55 mg/dL, administering of the active agent is reduced or even ceased completely. In some embodiments, if a subject's blood glucose level drops below a certain level, such as 70 ml/dL, the method further includes administering a bolus of dextrose solution to the subject. In one embodiment, the bolus can be a 5 wt % to 60 wt % dextrose solution in water or saline and may specifically be 50% dextrose in water (D50W), ½ normal saline, or normal saline.

In one aspect, the present disclosure provides a method for treating a subject at risk for CNS contusion expansion according to the above-described aspects, further involving measuring the degree of disability or dependence in the daily activities of the subject using the modified Rankin Scale (mRS) at 90 days and/or 180 days after completion of administration of the glyburide, a pharmaceutically acceptable salt thereof, variant or derivative thereof. The modified Rankin Scale (mRS) measures the degree of disability or dependence in the daily activities of participants who have suffered a stroke or other causes of neurological disability. The scale runs from 0-6, ranging from perfect health without symptoms (0) to dead (6). In one aspect, the present disclosure provides a method for treating a subject at risk for CNS contusion expansion according to the above-described aspects, wherein the risk of a mRS score of 4-6 in the subject is reduced by at least 20-70%, 30-60%, or 40-55% compared to subjects not treated with the method of the present disclosure.

In one aspect, the present disclosure provides a method for treating a subject at risk for CNS contusion expansion according to the above-described aspects, further involving measuring the Glasgow Outcome Scale (GOS) or Extended GOS (GOS-E) of the subject at 90 days and/or 180 days after completion of administration of the glyburide, a pharmaceutically acceptable salt thereof, variant or derivative thereof. The Glasgow Outcome Scale (GOS) is a global scale for functional outcome that rates participant status into one of five categories: Death (1), Vegetative State (2), Severe Disability (3), Moderate Disability (4) or Good Recovery (5). The Extended GOS (GOS-E) provides more detailed categorization into eight categories by subdividing the categories of severe disability, moderate disability and good recovery into a lower and upper category as follows: Death (1); Vegetative state (2); Lower severe disability (3); Upper severe disability (4); Lower moderate disability (5); Upper moderate disability (6); Lower good recovery (7); Upper good recovery (8). In one aspect, the present disclosure provides a method for treating a subject at risk for CNS contusion expansion according to the above-described aspects, wherein the risk of low GOS (1-4) or low GOS-E (1-5) of the subject is reduced by at least 20-70%, 30-60%, or 40-55% compared to subjects not treated with the method of the present disclosure.

In one aspect, the present disclosure provides a method for treating a subject at risk for CNS contusion expansion according to the above-described aspects, further involving performing delayed intubation if the subject suffers from neurologic deterioration during the treatment period. In one aspect, the delayed intubation is performed between 24 hours and 96 hours post-injury. In one aspect, the present disclosure provides a method for treating a subject at risk for CNS contusion expansion according to the above-described aspects, wherein the risk of delayed intubation of the subject is reduced by at least 20-70%, 30-60%, or 40-55% compared to subjects treated with conventional standard of care.

| | IV bolus | First Infusion | Second Infusion |
|---|---|---|---|
| Schedule 1 | 0.13 mg | 0.99 mg | 10.21 mg |
| | (22.5 mL over ~2 minutes) | (0.1644 mg/h for 6 h) | (0.1134 mg/h for 90 h) |
| Schedule 2 | 0.21 mg | 1.63 mg | 16.84 mg |
| | (37.6 mL over ~2 minutes) | (0.2722 mg/h for 6 h) | (0.1871 mg/h for 90 h) |

Additionally, a method of safely delivering glyburide, a pharmaceutically acceptable salt thereof, variant or derivative thereof, to a subject can include measuring liver enzyme levels while continuing administration of the glyburide, a pharmaceutically acceptable salt thereof, variant or derivative thereof. In certain aspects, steps can be taken to protect kidney, liver, intestines, or heart if the liver enzyme levels are abnormal, e.g., due to transient transaminase elevations. In some instances, the method further includes the preliminary step of measuring the subject's liver enzyme levels prior to administration. This can be done to establish a baseline liver enzyme level. The method can also include measuring liver enzyme levels at specified time periods after starting the administering step. In some embodiments, the subject's liver enzyme levels can be monitored at 4 hour intervals, 6 hour intervals, 8 hour intervals 12 hour intervals, 24 hour intervals, etc. Alternatively, levels can be checked at about 12 hours, about 24 hours, about 36 hours, about 48 hours, about 60 hours, about 72 hours, about 84 hours, about 96 hours, about 108 hours, about 120 hours, about 132 hours, about 154 hours, about 168 hours, the day the subject is discharged, or a combination thereof.

The monitoring can include measuring various enzymes. In one embodiment, the liver enzyme that is measured is aspartate transaminase (AST). In another embodiment, the liver enzyme that is measured is alanine transaminase (ALT). In another embodiment the liver enzyme is indirect, and/or direct, and/or total bilirubin. The monitoring can likewise involve measuring both the subject's AST and ALT values or ALT and bilirubin or ALT, AST and billirubin. In one embodiment, the administration of the glyburide, a pharmaceutically acceptable salt thereof, variant or derivative thereof, is discontinued if the subject's ALT or AST level rises to greater than about 8 times an upper limit of normal ALT or AST levels. In one embodiment, the administration of the SUR1-TRPM4 channel inhibitor can be discontinued if the subject's ALT or AST level rises to greater than about 6 times an upper limit of normal ALT or AST levels as determined by an administering practitioner. In yet another embodiment, the administration is discontinued if the subject's ALT or AST level rises to greater than about 4 times an upper limit of normal ALT or AST levels as determined by an administering practitioner. In a further embodiment, the administration of can be discontinued if the subject develops cholestatic jaundice or hepatitis. What these exact levels are and what constitutes an unsafe risk for continued treatment can be determined by the practitioner based on existing enzyme levels, levels considered to be safe, tradeoffs between enzyme levels vs. treating the CNS edema, etc. In one embodiment, the administration can be discontinued if the subject's total bilirubin level rises to greater than about 2 times an upper limit of normal.

The glyburide active, administration type, administration rate, administration time frame, administration time period for the first dose, underlying conditions, other details, etc., can be as discussed above and elsewhere herein, or can be modified according to clinical decisions made by the medical professional managing the treatment, for example. For example, different dosages and timings can carried out, or different routes of administration can also be carried out.

In one aspect, the present disclosure provides a method for treating a subject at risk for CNS contusion expansion according to the above-described aspects, wherein the subject is not suffering from a stroke, e.g., severe ischemic stroke. In one aspect, the present disclosure provides a method for treating a subject at risk for CNS contusion expansion according to the above-described aspects, wherein the subject is not suffering from large hemispheric infarction (LHI). In one aspect, the present disclosure provides a method for treating a subject at risk for CNS contusion expansion according to the above-described aspects, wherein the subject is not suffering from subarachnoid hemorrhage.

The present disclosure includes the following items and any and all combinations of elements, steps, and processes listed in the following list of items:
Item 1: A method of inhibiting expansion of cerebral contusion in a subject in need thereof, comprising administering a SUR1-TRPM4 channel inhibitor alone or in combination with one or more drugs or agents to a subject according to a predetermined dosing regimen to start within a predetermined time of initial contusion to the brain, spinal cord or other part of the CNS.
Item 2: A method for inhibiting secondary hemorrhage and capillary fragmentation in the brain of a subject in need thereof, comprising administering a SUR1-TRPM4 channel inhibitor alone or in combination with one or more drugs or agents to a subject according to a predetermined dosing regimen to start within a predetermined time of initial contusion to the brain, spinal cord or other part of the CNS.
Item 3: A method for reducing pericontusional edema and hemorrhage size in the brain of a subject in need thereof, comprising administering a SUR1-TRPM4 channel inhibitor alone or in combination with one or more drugs or agents to a subject according to a predetermined dosing regimen to start within a predetermined time of initial contusion to the brain, spinal cord or other part of the CNS.
Item 4: A method for decreasing water content in a CNS tissue of a subject in need thereof after CNS contusion, comprising administering a SUR1-TRPM4 channel inhibitor alone or in combination with one or more drugs or agents to a subject according to a predetermined dosing regimen to start within a predetermined time of initial contusion to the brain, spinal cord or other part of the CNS.
Item 5: A method for inhibiting disruption of the blood-brain barrier of a subject in need thereof after a CNS contusion, comprising administering a SUR1-TRPM4 channel inhibitor alone or in combination with one or more drugs or agents to a subject according to a predetermined dosing regimen to start within a predetermined time of initial contusion to the brain, spinal cord or other part of the CNS.
Item 6: A method for inhibiting CNS contusion progression and improving post-contusion motor function of a subject in need thereof, comprising administering a SUR1-TRPM4 channel inhibitor alone or in combination with one or more drugs or agents to a subject according to a predetermined dosing regimen to start within a predetermined time of initial contusion to the brain, spinal cord or other part of the CNS.
Item 7: A method for inhibiting microvascular impairment caused by endothelial cell swelling and fragmentation of a subject in need thereof, comprising administering a SUR1-TRPM4 channel inhibitor alone or in combination with one or more drugs or agents to a subject according to a predetermined dosing regimen to start within a predetermined time of initial contusion to the brain, spinal cord or other part of the CNS.
Item 8: A method for inhibiting extravasation of blood into the brain parenchyma of a subject in need thereof, comprising administering a SUR1-TRPM4 channel inhibitor alone or in combination with one or more drugs or agents to a subject according to a predetermined dosing regimen to start within a predetermined time of initial contusion to the brain, spinal cord or other part of the CNS.
Item 9: A method for inhibiting endothelial cell breakdown in a brain tissue of a subject in need thereof, comprising administering a SUR1-TRPM4 channel inhibitor alone or in combination with one or more drugs or agents to a subject according to a predetermined dosing regimen to start within a predetermined time of initial contusion to the brain, spinal cord or other part of the CNS.
Item 10: A method for inhibiting extravasation of endovascular fluids into the brain's interstitium of a subject in need thereof, comprising administering a SUR1-TRPM4 channel inhibitor alone or in combination with one or more drugs or agents to a subject according to a predetermined dosing regimen to start within a predetermined time of initial contusion to the brain, spinal cord or other part of the CNS.
Item 11: The method of any one or combination of items 1-10, wherein the predetermined dosing regimen is in an amount, at a rate, and for a duration to maintain a systemic steady state plasma level of the SUR1-TRPM4 channel inhibitor in a range of about 20-40 ng/ml, about 22-36 ng/ml, about 24-34 ng/mL, about 26-32 ng/mL, about 28-30 ng/mL, or about 25 ng/mL, about 26 ng/mL, about 27 ng/mL, about 28 ng/ml, about 29 ng/ml, about 30 ng/mL, about 31 ng/mL, or about 32 ng/mL, for a contusion-inhibiting treatment period.
Item 12: The method of any one or combination of item 1-11, wherein the predetermined time of initial contusion is within the first hour, first 2 hours, first 3 hours, first four 4 hours, first 6 hours, first 8 hours, or first 10 hours of the initial contusion to the subject's CNS tissue.
Item 13: The method of any one or combination of items 1-12, comprising administering about 3 mg to about 5.5 mg of the SUR1-TRPM4 channel inhibitor on day 1, about 2.5 mg to about 5 mg of the SUR1-TRPM4 channel inhibitor on day 2, about 2.5 mg to about 5 mg of the SUR1-TRPM4 channel inhibitor on day 3, and about 2.5 mg to about 5 mg of the SUR1-TRPM4 channel inhibitor on day 4.
Item 14: The method of any one or combination of items 1-12, comprising administering about 3-3.5 mg of the SUR1-TRPM4 channel inhibitor on day 1, and about 2.5-2.8 of the SUR1-TRPM4 channel inhibitor on days 2-4.
Item 15: The method of any one or combination of items 1-12, comprising administering about 5-5.5 mg of the SUR1-TRPM4 channel inhibitor on day 1, and about 4.2-4.8 mg of the SUR1-TRPM4 channel inhibitor on days 2-4.
Item 16: The method of any one of items 1-15, comprising administering an initial bolus injection of the SUR1-TRPM4 channel inhibitor on day 1 by a slow intravenous push, wherein the slow intravenous push is performed over a period of about 60 to 300 seconds, about 90 to 180 seconds, or about 120 seconds.
Item 17: The method of any one or combination of items 1-16, comprising administering an initial bolus injection of about 0.1 to about 0.2 mM of the SUR1-TRPM4 channel inhibitor, about 0.11 to about 0.15 mM of the SUR1-TRPM4 channel inhibitor, or about 0.11 to about 0.12 mM of the SUR1-TRPM4 channel inhibitor.
Item 18: The method of any one or combination of items 1-17, comprising administering an initial bolus injection having a volume of about 20 to 40 mL, about 22 to 38 mL, about 22 to 25 mL, or about 35 to 40 mL.
Item 19: The method of any one or combination of items 1-18, comprising administering about 0.1 to 0.3 mg of the SUR1-TRPM4 channel inhibitor, about 0.12 to 0.25 mg of the SUR1-TRPM4 channel inhibitor, or about 0.13 to 0.18 mg of the SUR1-TRPM4 channel inhibitor.
Item 20: The method of any one or combination of items 1-19, comprising administering a high dose continuous infusion for a period of about 4 to about 10 hours, about 6 to about 8 hours, about 5, or about 6 or about 7 hours in an amount sufficient to maintain a systemic steady state plasma level of 20-40 ng/mL, wherein the high dose continuous infusion comprises an aqueous solution of about 0.001 to about 0.1 mM of the SUR1-TRPM4 channel inhibitor, and wherein the high dose continuous infusion comprises administering 0.8 mg to about 2 mg of the SUR1-TRPM4 channel inhibitor to the subject.
Item 21: The method of item 20, wherein the high dose continuous infusion comprises an aqueous solution of about 0.005 mM to about 0.5 mM glyburide or about 0.005 to about 0.01 mM of the SUR1-TRPM4 channel inhibitor.
Item 22: The method of item 21, wherein the high dose continuous infusion comprises administering about 0.9 to about 1.7 mg, about 1 to about 1.7 mg, about 1 mg glyburide, or about 1.6 mg of the SUR1-TRPM4 channel inhibitor.
Item 23: The method of any one or combination of items 1-22, comprising administering a high dose continuous infusion at a rate of about 0.15 mg per hour to about 0.3 mg per hour, about 0.16 mg per hour to about 0.28 mg per hour, about 0.16 mg per hour to about 0.18 mg per hour, about 0.25 mg per hour to about 0.28 mg per hour, about 0.164 mg per hour, about 0.272 mg per hour of the SUR1-TRPM4 channel inhibitor.
Item 24: The method of any one or combination of items 1-23, comprising administering a low dose infusion in an amount of about 8- to 12-fold higher than the amount of the SUR1-TRPM4 channel inhibitor administered in a high dose infusion.
Item 25: The method of any one or combination of items 1-24, comprising administering a low dose infusion in an amount of about 75- to 100-fold higher or about 78- to 80-fold higher than the amount of the SUR1-TRPM4 channel inhibitor administered in an initial bolus administration of the SUR1-TRPM4 channel inhibitor.
Item 26: The method of any one or combination of items 1-25, comprising administering a high dose infusion in an amount of about 7- to 10-fold higher or about 7.5- to 8-fold than the amount of the SUR1-TRPM4 channel inhibitor administered in an initial bolus administration of the SUR1-TRPM4 channel inhibitor.
Item 27: The method of any one or combination of items 1-26, comprising administering the SUR1-TRPM4 channel inhibitor in an initial bolus phase, a high dose continuous infusion phase, and a low dose continuous infusion phase, wherein the SUR1-TRPM4 channel inhibitor is administered in a weight ratio of about 0.1-0.13:0.9-1.1:10-10.5 between the bolus phase, the high dose continuous infusion phase, and the low dose continuous infusion phase, respectively.
Item 28: The method of any one or combination of items 1-27, comprising administering the SUR1-TRPM4 channel inhibitor in a low dose infusion rate of about 20- to 25-fold lower or about 23- to 24-fold lower than the amount of the SUR1-TRPM4 channel inhibitor administered in an initial bolus administration of the SUR1-TRPM4 channel inhibitor.
Item 29: The method of any one or combination of items 1-28, comprising administering the SUR1-TRPM4 channel inhibitor in a low dose infusion rate of about 1.2- to 1.5-fold lower or about 1.4- to 1.5-fold lower than the amount of the SUR1-TRPM4 channel inhibitor f administered in a high dose continuous infusion phase of the SUR1-TRPM4 channel inhibitor.
Item 30: The method of any one or combination of items 1-29, comprising administering the SUR1-TRPM4 channel inhibitor in a high dose infusion rate of about 23 to 24-fold lower than the amount of the SUR1-TRPM4 channel inhibitor administered in an initial bolus administration of the SUR1-TRPM4 channel inhibitor.
Item 31: The method of any one or combination of items 1-30, comprising the SUR1-TRPM4 channel inhibitor in an initial bolus phase, a high dose continuous infusion phase, and a low dose continuous infusion phase, wherein the SUR1-TRPM4 channel inhibitor is administered in an hourly administration rate ratio of about 3.0-4.0:0.12-0.18:0.1-0.15 between the bolus phase, the high dose continuous infusion phase, and the low dose continuous infusion phase, respectively.

Item 32: The method of any one or combination of items 1-31, wherein a low dose continuous infusion of the SUR1-TRPM4 channel inhibitor is administered for a period of about 80 to about 120 hours, about 85 to about 100 hours, about 88, about 90 or about 92 hours, in an aqueous solution of about 0.001 to about 0.1 mM, about 0.005 mM to about 0.5 mM, or about 0.005 to about 0.01 mM of the SUR1-TRPM4 channel inhibitor.
Item 33: The method of item 32, comprising administering about 8 to about 20 mg of the SUR1-TRPM4 channel inhibitor in the low dose continuous infusion.
Item 34: The method of any one or combination of items 32 and 33, wherein 0.10 mg per hour to about 0.2 mg per hour, about 0.11 mg per hour to about 0.19 mg per hour, about 0.11 mg per hour to about 0.12 mg per hour, or about 0.18 mg per hour to about 0.19 mg per hour of the SUR1-TRPM4 channel inhibitor is administered to the subject.
Item 35: The method of any one or combination of items 1-34, wherein a total of 10-20 mg or about 17-20 mg of the SUR1-TRPM4 channel inhibitor is administered to the subject over about 96 hours.
Item 36: A method for decreasing vasogenic edema as measured by T2 flair magnetic resonance imaging in a subject, comprising:
   administering about 18-20 mg of a SUR1-TRPM4 channel inhibitor over 96 hours, the administering comprising a bolus injection of at least 5 mg of a SUR1-TRPM4 channel inhibitor, the bolus injection followed by a high dose continuous infusion, and the high dose continuous infusion followed by a low dose continuous infusion, wherein the high dose and low dose continuous infusions are administered at hourly infusion rate ratios of 1.2 to 1.7, 1.3 to 1.6, or 1.4 to 1.5 for the high dose infusion to the low dose infusion, wherein the low dose infusion is administered for 80 to 94 hours, 86 to 92 hours, or 90 hours; and
   maintaining a systemic steady state plasma level of 32-40 ng/ml in the subject.
Item 37: A method for decreasing matrix metalloprotease concentration in a CNS tissue of a subject, comprising:
   administering about 18-20 mg of a SUR1-TRPM4 channel inhibitor over 96 hours, the administering comprising a bolus injection of at least 5 mg of a SUR1-TRPM4 channel inhibitor, the bolus injection followed by a high dose continuous infusion, and the high dose continuous infusion followed by a low dose continuous infusion, wherein the high dose and low dose continuous infusions are administered at hourly infusion rate ratios of 1.2 to 1.7, 1.3 to 1.6, or 1.4 to 1.5 for the high dose infusion to the low dose infusion, wherein the low dose infusion is administered for 80 to 94 hours, 86 to 92 hours, or 90 hours; and
   maintaining a systemic steady state plasma level of 32-40 ng/mL in the subject.
Item 38: The method of any one or combination of items 1-37, comprising co-administering the SUR1-TRPM4 channel inhibitor with a sugar, a sugar alcohol or a combination thereof at a ratio of 1:10 to 1:1000, 1:20 to 1:500, 1:25 to 1:100, or 1:30.
Item 39: The method of item 38, where the sugar or sugar alcohol is selected from the group consisting of allitol, arabitol, dextrose, dulcitol, erythritol, galactitol, glycol, glycerol, iditol, isomalt, lactitol, maltitol, mannitol, sorbitol, threitol, xylitol, and combinations thereof.
Item 40: The method of any one or combination of items 1-39, comprising co-administering the SUR1-TRPM4 channel inhibitor with mannitol at a weight ratio of 1:20 to 1:100, 1:25 to 1:50, or 1:30 to 1:40.
Item 41: A method for reducing risk of a subject exhibiting CNS contusion expansion at 96 hours after the initial CNS contusion, comprising administering a SUR1-TRPM4 channel inhibitor alone or in combination with one or more drugs or agents to a subject according to a predetermined dosing regimen to start within a predetermined time of initial contusion to the brain, spinal cord or other part of the CNS according to any one of items 1-40, wherein the risk of the subject exhibiting contusion expansion at hour 96 after treatment with the method of the present disclosure is at least 2- to 30-fold, 3-fold, 4-fold, 5-fold, 10-fold, or 15-fold lower than the risk of a subject exhibiting contusion expansion at hour 96 without said administering.
Item 42: The method of item 41, wherein the CNS contusion expansion is either ≥50% increase from baseline contusion volume and ≥4.0 mL absolute increase from baseline, or ≥10.0 mL total expansion from baseline.
Item 43: The method of any one or combination of items 1-42, further comprising obtaining one or more images of the subject's CNS tissue before administering the SUR1-TRPM4 channel inhibitor and after administering the SUR1-TRPM4 channel inhibitor.
Item 44: The method of item 43, wherein the one or more images are a non-contrast computed tomography (NCCT), MRI image, or both.
Item 45: The method of item 42, further comprising obtaining a non-contrast computed tomography (NCCT), MRI image, or both, at 12 hours, 24, hours, 36 hours, 48 hours, 60 hours, and/or 72 hours after initiation of the administering.
Item 46: The method of item 42, further comprising obtaining a non-contrast computed tomography (NCCT), MRI image, or both, within days, weeks, or months after the completion of the administering.
Item 47: The method of any one or combination of items 1-46, further comprising measuring the subject's blood glucose level.
Item 48: The method of item 47, further comprising reducing the administered dose of the SUR1-TRPM4 channel inhibitor, if the subject's blood glucose level is <55 mg/dl (˜3.0 mmol/L).
Item 49: The method of item 48, wherein the administered dose is reduced by 25-75%.
Item 50: The method of item 48, wherein the rate of administration of the SUR1-TRPM4 channel inhibitor is reduced to a reduced rate of 0.05 to 0.10 mg/hr.
Item 51: The method of any one or combination of items 47-50, further comprising re-testing the subject's blood glucose level, and increasing the dose of the SUR1-TRPM4 channel inhibitor, if the subject's blood glucose level is <80 mg/dL (˜4.4 mmol/L) if the subject's blood glucose level is <80 mg/dL for three consecutive readings.
Item 52: The method of item 51, wherein the reduced rate of the SUR1-TRPM4 channel inhibitor administered per hour is increased by 15-65%, 20-55%, or 30-50%.
Item 53: The method of any one or combination of items 1-52, further comprising measuring the degree of disability or dependence in the daily activities of the subject using the modified Rankin Scale (mRS) at 90 days and/or 180 days after completion of the administering.
Item 54: The method of any one or combination of items 1-53, further comprising measuring the Glasgow Outcome Scale (GOS) or Extended GOS (GOS-E) of the subject at 90 days and/or 180 days after completion of the administering.
Item 55: The method of any one or combination of items 1-54, wherein the SUR1-TRPM4 channel inhibitor is selected from the group consisting of glyburide, 4-trans-hydroxy-glibenclamide, 3-cis-hydroxyglibenclamide, tolbutamide, chlorpropamide, tolazamide, repaglinide, nateglinide, meglitinide, midaglizole, gliquidone, LY397364, LY389382, gliclazide, glimepiride, metabolites that interact with SUR1, salts, and combinations thereof.
Item 56: The method of any one or combination of items 1-54, wherein the SUR1-TRPM4 channel inhibitor is glyburide or a pharmaceutically acceptable salt thereof.

### Example 1: Randomized, Double Blind, Placebo-Controlled Study in Patients with Brain Contusion

In order to determine the efficacy of glyburide, a pharmaceutically acceptable salt thereof, variant or derivative thereof for reducing or treating progression of CNS contusion in patients with brain contusion, a randomized double blind placebo controlled clinical study is conducted. Approximately 160 patients between ages 18 to 80 years, with a diagnosis of brain contusion, with lesions within the supratentorial brain parenchyma totaling >3 mL in volume per Investigator assessment of baseline non-contrast computed tomography scan, are randomized. Participants have a score of 5 to 14 on the Glasgow Coma Scale (GCS) and have been functionally independently prior to the brain contusion.

All participants are dosed within 6.5 hours of time of trauma/last known normal, and receive study treatment (3 mg/day or 5 mg/day) or matching placebo administered as a 3-stage continuous infusion: a bolus IV dose (given over 2 minutes), followed by 2 different infusion rates (a rapid IV infusion for 6 hours followed by a slow IV infusion for the remaining 90 hours) for 96 hours total as follows:

| | IV bolus | First Infusion | Second Infusion |
|---|---|---|---|
| Schedule 1 | 0.13 mg | 0.99 mg | 10.21 mg |
| | (22.5 mL over ~2 minutes) | (0.1644 mg/h for 6 h) | (0.1134 mg/h for 90 h) |
| Schedule 2 | 0.21 mg | 1.63 mg | 16.84 mg |
| | (37.6 mL over ~2 minutes) | (0.2722 mg/h for 6 h) | (0.1871 mg/h for 90 h) |

Participants are randomly assigned to 3 mg/day study treatment, 5 mg/day study treatment, 3 mg/day matching placebo, or 5 mg/day matching placebo in a 1:1:1:1 ratio, stratified based on baseline contusion volume per Investigator assessment of non-contrast computed tomography scan or site local radiologic assessment (3 to 10 mL or >10 mL), baseline GCS (5 to 8, 9 to 12, or 13 to 14), age (≤70 years old or >70 years old), and region (North America [United States and Canada] or Rest of World).

The study treatment or matching placebo is administered for 96 hours (4 days) in total. All participants will be followed up for functional outcome measures at Day 90 and Day 180.

Measurements of contusion expansion are based on a comparison of the baseline images and the 96-hour scan or the scan obtained prior to any neurosurgical intervention (NSx) or comfort measures only (CMO). NSx includes craniotomy and decompressive craniectomy (DC).

The primary endpoint is the proportion of participants with contusion (hematoma plus perihematomal edema) expansion by Hour 96 (or prior to NSx or CMO). To meet criteria for expansion of total contusion volume per the central review, a participant must have the following: ≥50% increase from baseline contusion volume, and ≥4.0 mL absolute increase from baseline OR≥10.0 mL total expansion from baseline.

Secondary endpoints include the effects on acute neurologic status, functional outcomes, and treatment requirements are evaluated. The participants are tested according to the Glasgow Outcome Scale-Extended (GOS-E) at Day 90 and Day 180. The participants are tested according to the Modified Rankin Scale (mRS) at Day 90 and Day 180. The proportion of participants requiring delayed intubation is recorded. Delayed intubation is defined as participants requiring intubation (for neurologic deterioration only) at any time between 24 hours and 96 hours postinjury.

A shift analysis (p-value obtained using 2-sided Mann Whitney test, and a common odds ratio [OR] estimated under a proportional odds model) is expected to find improvement across the mRS in favor of the method of the present disclosure compared to control at 90 and 180 days.

Secondary endpoints include the differential effects on hematoma and edema expansion are examined to further differentiate the mechanism of action of glyburide on contusion expansion. Data on the change in absolute hematoma volume from baseline to 24 hours; change in absolute edema volume from baseline to 96 hours is collected.

Another secondary endpoint includes the incidence of decompressive craniectomy in the individuals, which is recorded at 90 and 180 days following treatment.

Another secondary endpoint includes the incidence of mortality in the individuals, which is recorded at 90 and 180 days following treatment.

It is expected that the primary endpoint and one or more of the secondary endpoints will be significantly higher or improved when using the method of the present disclosure compared to placebo. Further, it is expected that the method will inhibit secondary hemorrhage and capillary fragmentation in the brain, will reduce pericontusional edema and hemorrhage size in the brain, will decrease water content in a CNS tissue, will inhibit disruption of the blood-brain barrier, will inhibit CNS contusion progression and improving post-contusion motor function, will inhibit microvascular impairment caused by endothelial cell swelling and fragmentation, will inhibit extravasation of blood into the brain parenchyma of a subject, will inhibit endothelial cell breakdown in a brain tissue, will inhibit extravasation of endovascular fluids into the brain's interstitium, will decrease vasogenic edema as measured by T2 flair magnetic resonance imaging, and will decrease matrix metalloprotease concentration in the subject's CNS tissue.

The participants further undergo the Rey auditory and visual learning test (RAVLT), a functional status examination, liver function test, electrocardiogram, and plasma biomarker assessments including for MMP-9.

### Example 2: Randomized, Double Blind, Placebo-Controlled Study in Traumatic Brain Injury

A double-blind, placebo-controlled study to assess the effects of glyburide, as a representative SUR1-TRPM4 channel inhibitor, on edema in TBI was conducted at 3 clinical sites involving 29 subjects with moderate or severe TBI (Glasgow Coma Scale score 4-12) or with mild TBI (GCS 13-14) having hemorrhage on CT scan. The administration of the test drug was as follows: a bolus of 0.13 mg of the SUR1-TRPM4 channel inhibitor was administered over approximately 2 minutes followed by a continuous infusion of 0.16 mg/hr for 6 hours, and then a continuous infusion of 0.11 mg/hr for 66 hours, for a total dosing period of 72 hours. The total daily dose of Study Drug on Day 1, Day 2 and Day 3 was 3.12 mg, 2.67 mg, and 2.67 mg, respectively. All subjects were assessed by MRI scans at baseline and 72 hours.

Inclusion criteria included closed head TBI and a time of injury no more than 10 hours before drug administration. Exclusion criteria included: penetrating brain injury; spinal column instability and/or spinal cord injury; concomitant severe non survivable injury; International Normalized Ratio >1.4; systolic blood pressure <90 mm Hg not responsive to fluid resuscitation; hospitalization for brain injury, psychiatric or neurological disease within previous 3 years; surgical operation anticipated that would prevent dosing with study drug within 10 hours of injury; and oxygen partial pressure <60 mm Hg.

The primary finding of the study was based on MRI and utilized a free water analysis. In the free water analysis, the data originating from each individual voxel is modelled as a mixture of two components. The first component (the free water compartment) consists of water molecules which are free to diffuse, i.e., during the experiment time the molecules are not reflected, hindered or constrained by their surroundings, and are hence free and have the diffusion coefficient (speed) expected for water. The second component (the tissue compartment) is composed of all other water molecules within the voxel, typically these are water molecules that are within or between cellular components of the tissue, which constrain or hinder molecule motion.

The relative volume of the free water compartment in the voxel, which does not have units, ranges between 0 and 1, where FW of 0 means that there are no molecules that were found to belong to the free water compartment, and FW of 1 means that 100% of the water molecules within the voxel are found to belong to the free water compartment. Previous findings indicate that vasogenic edema has similar diffusion properties as free water, hence, fitting the free water model to vasogenic edema is expected to yield very similar results: FW is expected to be significantly higher than 0 on voxels within the edema, and the values of the FW would describe how much of the voxel is occupied by the edema. For example, if a voxel has 40% edema and 60% tissue, the FW parameter is expected to be 0.4.

Based on an assessment of the principal investigator, a population of lesioned patients—those with evidence of blood at baseline—was defined and analyzed. The 14 subjects (7 drug and 7 placebo) identified as having evidence of lesional blood (i.e., blood associated with a contusion) at baseline and having MRI scans at both baseline and 72 hours were assessed for edema using MIM software version 5.6. Blinded readers ascertained x, y, z measurements of the total apparent contusion using T2 images, unless missing, in which case, DTI images were used. Only contusions that could be reliably measured (measuring at least about 1 cc) were measured. Readers also ascertained the volume measurements of hemorrhages in a similar manner using SWI images. Linear measurements were captured in centimeters (cm) and all volumes in cubic centimeters (cc). A blinded reader, using the ABC/2 method, assessed the size of the contusion alone and the size of the contusion including any peri-contusional edema. The size of the contusion was subtracted out to obtain the volume of the edema. In subjects with multiple lesions, the volume of all lesions was added to obtain an overall edema volume.

The drug group at baseline had a larger mean volume of edema (8.72 ml versus 2.12 ml for placebo, a 4-fold difference) and worse clinical characteristics than the placebo group, as measured by the mean motor and eye domains of the Glasgow Coma Scale (GCS; 6.14 versus 7.67 in the placebo) and the rate of intubation (5 of 7 versus 3 of 7 in placebo). Despite this worse clinical condition of patients in the drug group, the drug group showed less evolution of edema from baseline to 72 hours (2.14-fold growth versus 10.28-fold growth in placebo) and the drug group showed no worsening in the mean Glasgow Coma Scale Extended score at 180 days, whereas the placebo group showed worsening (GOSE; 6 versus 6.17 for placebo). It can be concluded from these data that, despite a more severe clinical profile and 4-fold more edema at baseline than placebo, glyburide substantially reduced the expansion or growth of edema. No drug-related effects were detected in non-lesioned subjects or in areas of the brain without a lesion, indicating the drug effect is surprisingly, not edema-specific, but specific to edema associated with a lesion.

It can be expected, therefore, that in a study consisting exclusively of subjects identified at baseline as having a radiologically-defined (e.g., CT or MRI) lesion that can be reliably measured (approximately 0.5 cc or more) and balanced in terms of baseline clinical characteristics, such as the GCS or its individual components (motor, eye, verbal), that the drug group would result in improved clinical outcomes on scales such as the GOSE with a minimum effect size of at least 4% or an odds ratio of at least 1.2, favoring drug over placebo.

Any of the above protocols or similar variants thereof can be described in various documentation associated with a pharmaceutical product. This documentation can include, without limitation, protocols, statistical analysis plans, investigator brochures, clinical guidelines, medication guides, risk evaluation and mediation programs, prescribing information and other documentation that may be associated with a pharmaceutical product. It is specifically contemplated that such documentation may be physically packaged with a SUR1-TRPM4 channel inhibitor pharmaceutical product as a kit, as may be beneficial or as set forth by regulatory authorities.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

We claim:

1. A method of inhibiting expansion of cerebral contusion in a subject in need thereof or inhibiting secondary hemorrhage and capillary fragmentation in the subject's brain or reducing pericontusional edema and hemorrhage size in the subject's brain, comprising administering a SUR1-TRPM4 channel inhibitor in combination with mannitol at a weight ratio of 1:20 to 1:100 to the subject according to a predetermined dosing regimen to start within a predetermined time of initial contusion to the brain of the subject.

2. The method of claim 1, wherein the predetermined dosing regimen is in an amount, at a rate, and for a duration to maintain a systemic steady state plasma level of the SUR1-TRPM4 channel inhibitor in a range of about 20-40 ng/mL for a contusion-inhibiting treatment period.

3. The method of claim 1, wherein the predetermined time of initial contusion is within the first 10 hours of the initial contusion.

4. The method of claim 1, comprising administering about 3 mg to about 5.5 mg of the SUR1-TRPM4 channel inhibitor on day 1, about 2.5 mg to about 5 mg of the SUR1-TRPM4 channel inhibitor on day 2, and about 2.5 mg to about 5 mg of the SUR1-TRPM4 channel inhibitor on day 3.

5. The method of claim 1, comprising administering about 3-3.5 mg of the SUR1-TRPM4 channel inhibitor on day 1, and about 2.5-2.8 of the SUR1-TRPM4 channel inhibitor on days 2-3.

6. The method of claim 1, comprising administering about 5-5.5 mg of the SUR1-TRPM4 channel inhibitor on day 1, and about 4.2-4.8 mg of the SUR1-TRPM4 channel inhibitor on days 2-3.

7. The method of claim 1, comprising administering an initial bolus injection of the SUR1-TRPM4 channel inhibitor on day 1 by a slow intravenous push, wherein the slow intravenous push is performed over a period of about 60 to 300 seconds, wherein the initial bolus injection is about 0.1 to about 0.2 mM of the SUR1-TRPM4 channel inhibitor, and wherein the initial bolus injection has a volume of about 20 to 40 mL.

8. The method of claim 1, comprising administering a high dose continuous infusion for a period of about 4 to about 10 hours in an amount sufficient to maintain a systemic steady state plasma level of 20-40 ng/ml, wherein the high dose continuous infusion comprises an aqueous solution of about 0.001 to about 0.1 mM of the SUR1-TRPM4 channel inhibitor, and wherein the high dose continuous infusion comprises administering 0.8 mg to about 2 mg of the SUR1-TRPM4 channel inhibitor to the subject.

9. The method of claim 8, wherein the high dose continuous infusion comprises an aqueous solution of about 0.005 mM to about 0.5 mM glyburide, and wherein the high dose continuous infusion comprises administering about 0.9 to about 1.7 mg glyburide.

10. The method of claim 1, comprising administering a high dose continuous infusion at a rate of about 0.15 mg per hour to about 0.3 mg per hour of the SUR1-TRPM4 channel inhibitor.

11. The method of claim 9, comprising administering a low dose infusion in an amount of about 8- to 12-fold higher than the amount of the SUR1-TRPM4 channel inhibitor administered in the high dose infusion.

12. The method of claim 7, comprising administering a low dose infusion in an amount of about 75- to 100-fold higher than the amount of the SUR1-TRPM4 channel inhibitor administered in the initial bolus administration of the SUR1-TRPM4 channel inhibitor.

13. The method of claim 7, comprising administering a high dose infusion in an amount of about 7- to 10-fold higher or about 7.5- to 8-fold than the amount of the SUR1-TRPM4 channel inhibitor administered in the initial bolus administration of the SUR1-TRPM4 channel inhibitor.

14. The method of claim 1, comprising administering the SUR1-TRPM4 channel inhibitor in an initial bolus phase, a high dose continuous infusion phase, and a low dose continuous infusion phase, wherein the SUR1-TRPM4 channel inhibitor is administered in an hourly administration rate ratio of about 3.0-4.0:0.12-0.18:0.1-0.15 between the bolus phase, the high dose continuous infusion phase, and the low dose continuous infusion phase, respectively.

15. The method of claim 1, comprising co-administering the SUR1-TRPM4 channel inhibitor with mannitol at a weight ratio of 1:25 to 1:50.

16. The method of claim 1, further comprising measuring the degree of disability or dependence in the daily activities of the subject using the modified Rankin Scale (mRS) at 90 days and/or 180 days after completion of the administering.

17. The method of claim 1, further comprising measuring the Glasgow Outcome Scale (GOS) or Extended GOS (GOS-E) of the subject at 90 days and/or 180 days after completion of the administering.

18. The method of claim 1, wherein the SUR1-TRPM4 channel inhibitor is selected from the group consisting of glyburide, 4-trans-hydroxy-glibenclamide, 3-cis-hydroxyglibenclamide, tolbutamide, chlorpropamide, repaglinide, nateglinide, meglitinide, midaglizole, tolazamide, gliquidone, LY397364, LY389382, gliclazide, glimepiride, metabolites that interact with SUR1, salts, and combinations thereof.

19. The method of claim 1, wherein the SUR1-TRPM4 channel inhibitor is glyburide or a pharmaceutically acceptable salt thereof.

20. The method of claim 1, wherein the subject has brain contusion within the supratentorial brain parenchyma totaling >3 mL in volume and a score of 5 to 14 on the Glasgow Coma Scale (GCS).
